(19) United States
(12) Patent Application Publication
(10) Pub. No.: US 2025/0195506 A1
(43) Pub. Date: Jun. 19, 2025

(54) COMBINATION THERAPEUTICS FOR ANTIVIRAL TREATMENT

(71) Applicant: SELVA THERAPEUTICS, INC., San Mateo, CA (US)

(72) Inventors: Felix W. FRUEH, San Mateo, CA (US); Daniel C. MANEVAL, Del Mar, CA (US)

(73) Assignee: SELVA THERAPEUTICS, INC., San Mateo, CA (US)

(21) Appl. No.: 18/976,108

(22) Filed: Dec. 10, 2024

Related U.S. Application Data
(60) Provisional application No. 63/609,966, filed on Dec. 14, 2023.

(51) Int. Cl. A61K 31/495; A61K 31/706; A61K 31/7068

(52) U.S. Cl. CPC A61K 31/495; A61K 31/706; A61K 31/7068

(57) ABSTRACT

The present application relates to viral entry inhibitors and RNA polymerase inhibitors, pharmaceutical compositions comprising one or more of the same, and combination therapies, as well as methods of making and of using the foregoing in the treatment of certain diseases.

### INCORPORATION BY REFERENCE OF RELATED APPLICATIONS

This application is based upon and claims priority under 35 USC 119(e) to U.S. Provisional Application No. 63/609,966 filed Dec. 14, 2023, the entire contents of all of which are incorporated herein by reference in their entirety.

### FIELD OF THE INVENTION

The present application relates to antiviral compounds and combinations designed to prevent or treat viral infections, pharmaceutical compositions comprising the foregoing, and methods of making and of using the foregoing in the treatment of certain diseases.

### BACKGROUND

Viral diseases continue to pose a significant risk to public health. For example, Influenza viruses, Filoviruses (like Ebola virus), Coronaviruses (like SARS-COV and MERS-COV), and Henipaviruses are highly pathogenic viruses which have caused, or threaten to cause, major outbreaks. There is currently a need for antiviral therapeutics designed to prevent or treat viral diseases. Indeed, broad-spectrum antiviral drugs, with overlapping therapeutic indications, would facilitate rapid responses to viral threats, potentially even without precise identification of the agent

### SUMMARY

Provided are antiviral compounds and combinations designed to prevent or treat viral infections, pharmaceutical compositions comprising the foregoing, and methods of making and of using the foregoing in the treatment of certain diseases.

The inventors of the present application have developed viral entry inhibitors that block infection of a broad array of viruses. In particular, the viral entry inhibitors of the present application are cysteine protease inhibitors and serine protease inhibitors. Without being bound by theory, the viral entry inhibitors of the present application prevent viral entry into host cells by blocking activation of viral glycoproteins, in particular cysteine proteases and serine proteases.

Thus, in one aspect, the present application includes cysteine protease inhibitors. Preferably, the cysteine protease inhibitor is K11777 (SLV-213):

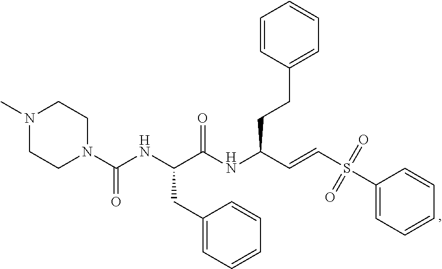 [SMILES: CN1CCN(C(=O)N[C@@H](Cc2ccccc2)C(=O)N[C@H](/C=C/S(=O)(=O)c2ccccc2)CCc2ccccc2)CC1]

and pharmaceutically acceptable salts thereof.

In another aspect, the present application includes serine protease inhibitors. Preferably, the serine protease inhibitor is camostat:

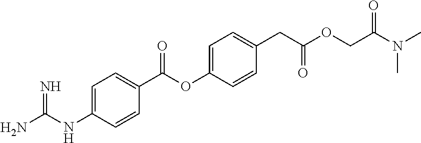 [SMILES: CN(C)C(=O)COC(=O)Cc1ccc(OC(=O)c2ccc(NC(=N)N)cc2)cc1]

and pharmaceutically acceptable salts thereof.

In another aspect, the present application includes nucleoside analogs and viral RNA polymerase inhibitors and prodrugs thereof. A preferable compound of this class is Remdesivir:

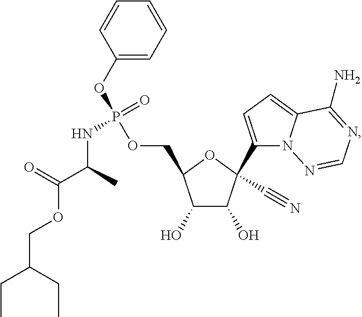 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

and pharmaceutically acceptable salts thereof.

In another aspect, the present application includes the combination of a viral entry inhibitor and a nucleoside analog, RNA polymerase inhibitor, or prodrug thereof. The inventors of the present application have found such combinations exhibit surprising and significant synergistic effects.

In another aspect, the present application provides pharmaceutical compounds and formulations for treatment of certain diseases comprising compounds of the invention and pharmaceutically acceptable excipients. The pharmaceutical formulations are adapted for various routes of administration.

Thus, in one aspect of the present application, the antiviral compounds, combinations, formulations, and methods of the present application are useful for treating viral infections, including viral infections which rely on activation by cellular proteases and viruses capable of incorporating nucleoside analogs (like Remdesivir and GS-441524) during replication. Such infections may be caused by Filoviruses (e.g., Ebola virus (EBOV) and Marburg virus (MARV)), Coronaviruses (e.g., SARS-COV-2, SARS-COV, MERS-COV), Influenza viruses (e.g., Influenzavirus A (H1N1, H7N9, H5N1, etc.), B, C, and D), Hepatitis viruses (e.g. Hepatitis A, B, C, D, E), Retroviruses (e.g., Lentiviruses including HIV), Herpesviruses (e.g. Varicella Zoster, Herpes simplex 1 and 2, etc.), Rhinoviruses, Rotaviruses, Human Papillomaviruses, Henipaviruses, Adenoviruses, Noroviruses, Rotaviruses, Sapoviruses, Astroviruses, Coxsackieviruses, Echoviruses, Enteroviruses, Orthomyxoviruses, Paramyxoviruses (e.g., Measles virus), Motonaviruses (e.g. Rubella viruses), Flaviviruses (e.g. Dengue viruses, Yellow fever viruses), Poxviruses (e.g., Variola virus), Alphaviruses (e.g., Chikungunya virus), Orthohantaviruses (e.g. Hanta virus), and Rabies viruses.

In another aspect, the present application provides methods of treatment of certain diseases, including those listed above, using compounds or pharmaceutical compositions discussed herein. Some methods of the present application relate to the treatment of certain patient subpopulations, which require special and/or different considerations when receiving treatment.

In another aspect, the present application provides methods of synthesizing or preparing compounds and pharmaceutical compositions discussed herein.

In another aspect, the present application provides kits containing the compounds or pharmaceutical formulations of the present application. Such kits may include a single agent according the present application or multiple agents. The agents may be packaged separately or together. Such kits may include prescribing information. Such kits may also include tests for rapidly identifying a patient infected by a pathogen or suffering from a disease so that treatment can be initiated quickly. Such kits may also include tests for identifying patient subpopulations which require special and/or different considerations when receiving treatment.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration by injection.

Further objects, features, and advantages of the present application will become apparent form the detailed description, which is set forth below when considered together with the figures of drawing.

### DETAILED DESCRIPTION OF CERTAIN EMBODIMENTS

### Definitions

“K11777” and “K777” are alternative designations for the same compound. SLV-213 and S-001 also refer to this compound.

“Remdesivir” is also referred to commercially as VEKLURY.

“Camostat” is also referred to commercially using several names, including Archiment, Camoent, Camostat Mesilate, Camopan, Camostate, Camoston, Camotat, Carmozacine, Foipan, Foipan Ilsung, Kamostaal, Leanac, Leseplon, Libilister, Mecilpan, Mospan, Pancrel, and Raintat.

In the context of the present application, the terms “about” and “approximately” mean any value that is within ±10% of the value referred to. The term “substantially” is used to indicate that a value is close to a targeted value, where close means within 90% of the targeted value.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

A “decrease” can refer to any change that results in a smaller amount of a symptom, disease, composition, condition, or activity. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage. In particular, the term “treatment” includes the alleviation, in part or in whole, of the symptoms of an infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of an infection in patients. However, it will be appreciated that such prevention may not be absolute, i.e., it may not prevent all such patients developing an infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, hamster, ferret, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

As used herein, the term “bioorthogonal” or “bioorthogonal functional group” refer to a functional group or chemical reaction that can occur inside a living cell, tissue, or organism without interfering with native biological or biochemical processes. A bioorthogonal functional group or reaction is not toxic to cells.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Unless otherwise stated, structures depicted herein are also meant to include all isomeric (e.g., enantiomeric, diastereomeric, and geometric (or conformational)) forms of the structure; for example, the R and S configurations for each stereocenter, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

If, for instance, a particular enantiomer of a compound of the present invention is desired, it may be prepared by asymmetric synthesis, chiral chromatography, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

One of ordinary skill in the art will appreciate that the synthetic methods, as described herein, utilize a variety of protecting groups. By the term “protecting group,” as used herein, it is meant that a particular functional moiety, e.g., O, S, or N, is masked or blocked, permitting, if desired, a reaction to be carried out selectively at another reactive site in a multifunctional compound. In preferred embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers); and the protecting group will preferably have a minimum of additional functionality to avoid further sites of reaction. As detailed herein, oxygen, sulfur, nitrogen, and carbon protecting groups may be utilized. By way of non-limiting example, hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy) methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)-amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N′,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described by Greene and Wuts (supra).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph, which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6-membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{Δ}, -(haloR^{Δ}), —(CH₂)₀₋₂OH, —(CH₂)₀₋₂OR^{Δ}, —(CH₂)₀₋₂CH(OR^{Δ})₂; —O(haloR^{Δ}), —CN, —N₃, —(CH₂)₀₋₂C(O)R^{Δ}, —(CH₂)₀₋₂C(O)OH, —(CH₂)₀₋₂C(O)OR^{Δ}, —(CH₂)₀₋₂SR^{Δ}, —(CH₂)₀₋₂SH, —(CH₂)₀₋₂NH₂, —(CH₂)₀₋₂NHR^{Δ}, —(CH₂)₀₋₂NR^{Δ}, —NO₂, —SiR^{Δ}₃, —OSiR^{Δ}₃, —C(O)SR^{Δ}, —(C₁₋₄ straight or branched alkylene)C(O)OR^{Δ}, or —SSR. wherein each R^{Δ} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{Δ}, -(haloR^{Δ}), —OH, —OR^{Δ}, —O(haloR^{Δ}), —CN, —C(O)OH, —C(O)OR^{Δ}, —NH₂, —NHR^{Δ}, —NR^{Δ}₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur. Suitable substituents on the aliphatic group of R^{†} are independently halogen, —R^{Δ}, -(haloR^{Δ}), —OH, —OR^{Δ}, —O(haloR^{Δ}), —CN, —C(O)OH, —C(O)OR^{Δ}, —NH₂, —NHR^{Δ}, —NR^{Δ}₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The term “enriched” as used herein refers to a mixture having an increased proportion of one or more species. In some embodiments, the mixture is “enriched” following a process that increases the proportion of one or more desired species in the mixture. In some embodiments, the desired species comprise(s) greater than 10% of the mixture. In some embodiments, the desired species comprise(s) greater than 25% of the mixture. In some embodiments, the desired species comprise(s) greater than 40% of the mixture. In some embodiments, the desired species comprise(s) greater than 60% of the mixture. In some embodiments, the desired species comprise(s) greater than 75% of the mixture. In some embodiments, the desired species comprise(s) greater than 85% of the mixture. In some embodiments, the desired species comprise(s) greater than 90% of the mixture. In some embodiments, the desired species comprise(s) greater than 95% of the mixture. Such proportions can be measured any number of ways, for example, as a molar ratio, volume to volume, or weight to weight.

The term “pure” refers to compounds that are substantially free of compounds of related non-target structure or chemical precursors (when chemically synthesized). This quality may be measured or expressed as “purity.” In some embodiments, a target compound has less than about 30%, 20%, 10%, 5%, 2%, 1%, 0.5%, and 0.1% of non-target structures or chemical precursors. In certain embodiments, a pure compound of present invention is only one prosapogenin compound (i.e., separation of target prosapogenin from other prosapogenins).

The term “carbohydrate” refers to a sugar or polymer of sugars. The terms “saccharide”, “polysaccharide”, “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

### Compounds

Compounds of this invention include those described generally above, and are further illustrated by the classes, subclasses, and species disclosed herein. For purposes of this invention, the chemical elements are identified in accordance with the Periodic Table of the Elements, CAS version, Handbook of Chemistry and Physics, 75th Ed. Additionally, general principles of organic chemistry are described in Organic Chemistry, Thomas Sorrell, University Science Books, Sausalito: 1999, and March's Advanced Organic Chemistry, 5th Ed., Ed.: Smith, M. B. and March, J., John Wiley & Sons, New York: 2001, the entire contents of which are hereby incorporated by reference.

### Viral Entry Inhibitors

In some embodiments, the provided compounds are viral entry inhibitors.

In some embodiments, the viral entry inhibitors are cysteine protease inhibitors. In some embodiments, the provided compounds include reversible and irreversible Cathepsin inhibitors. In some embodiments, the Cathepsin inhibitors are pan-Cathepsin inhibitors. In some embodiments, the cysteine protease inhibitors of the present application include peptidases from the Papain family, i.e. Family C1 and Clan CA in the MEROPS database classification, as well as their derivatives and analogues. In other embodiments, the cysteine protease inhibitor is K11777 (SLV-213) (also known as K777):

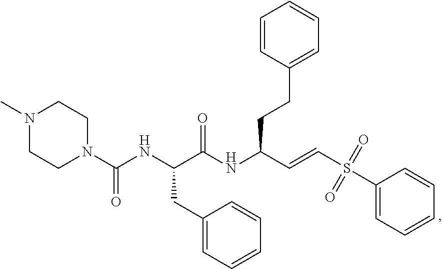 [SMILES: CN1CCN(C(=O)N[C@@H](Cc2ccccc2)C(=O)N[C@H](/C=C/S(=O)(=O)c2ccccc2)CCc2ccccc2)CC1]

or a pharmaceutically acceptable salt thereof.

In some embodiments, the provided compound is:

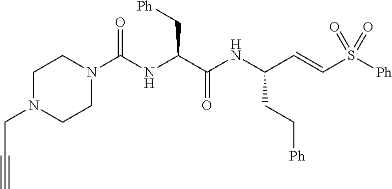 [SMILES: C#CCN1CCN(C(=O)N[C@@H](Cc2ccccc2)C(=O)N[C@H](/C=C/S(=O)(=O)c2ccccc2)CCc2ccccc2)CC1]

or a pharmaceutically acceptable salt thereof.

In other embodiments, the cysteine protease inhibitor includes K11777 derivates, including, for example, WRR-483, (S)-Benzyl 2-amino-5-(3-(2,2,4,6,7-pentamethyl-2,3-dihydro-benzofuran-5-ylsulfonyl)guanidino)pentanoate (4), (S)-Benzyl 2-(4-methylpiperazine-1-carboxamido)-5-(3-(2,2,4,6,7-pentamethyl-2,3-dihydrobenzofuran-5-ylsulfonyl)guanidino)pentanoate, (S)-2-(4-Methylpiperazine-1-carboxamido)-5-(3-(2,2,4,6,7-pentamethyl-2,3-dihydrobenzofuran-5-ylsulfonyl)guanidino)pentanoic acid (6), and 4-Methyl-N—((S)-1-oxo-5-(3-(2,2,4,6,7-pentamethyl-2,3-di-hydrobenzofuran-5-ylsulfonyl)guanidino)-1-((S,E)-5-phenyl-1-(phenylsulfonyl)pent-1-en-3-ylamino)pentan-2-yl)pipera-zine-1-carboxamide. In other embodiments, the cysteine protease inhibitor includes odanacatib, Cz001, Cz002, Cz003, Cz005, Cz007, or Cz008.

In some embodiments, the viral entry inhibitors are serine protease inhibitors. In some embodiments of the present application, the serine protease inhibitors are TMPRSS inhibitors, preferably TMPRSS2 inhibitors. In one embodiment, the serine protease inhibitor is camostat:

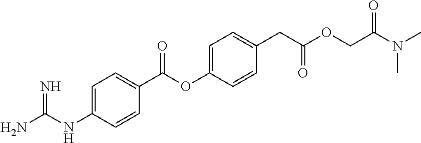 [SMILES: CN(C)C(=O)COC(=O)Cc1ccc(OC(=O)c2ccc(NC(=N)N)cc2)cc1]

or a pharmaceutically acceptable salt thereof. In another embodiment, the serine protease inhibitor is camostat mesylate. In another embodiment, the present application includes camostat derivates and their pharmaceutically acceptable salts.

### Nucleoside Analogs and RNA Polymerase Inhibitors

In some embodiments, the provided compounds are nucleoside analogs and/or RNA polymerase inhibitors. In some embodiments, the provided compound is a compound of Formula I:

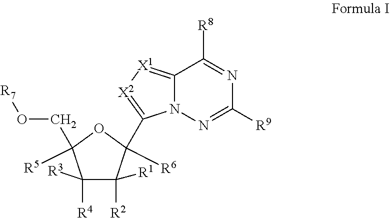

or a pharmaceutically acceptable salt, thereof;
wherein:
each R¹, R², R³, R⁴, or R⁴ is independently H, OR^{a}, N(R^{a})₂, N₃, CN, NO₂, S(O)ₙR^{a}, halogen, (C₁-C₈)alkyl, (C₄-C₈)carbocyclylalkyl, (C₁-C₈)substituted alkyl, (C₂-C₈)alkenyl, (C₂-C₃)substituted alkenyl, (C₂-C₈)alkynyl, (C₂-C₈)substituted alkynyl, or aryl(C₁-C₈)alkyl;
or any two R¹, R², R³, R⁴, or R⁶ on adjacent carbon atoms when taken together are —O(CO)O— or when taken together with the ring carbon atoms to which they are attached form a double bond;
R⁶ is OR^{a}, N(R^{a})₂, N₃, CN, NO₂, S(O)ₙR^{a}, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), —SO₂NR¹¹R¹², halogen, (C₁-C₈)alkyl, (C₄-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or aryl(C₁-C₈)alkyl or R⁶ and either R¹ or R² when taken together are —O(CO)O—;
each n is independently 0, 1, or 2;
each R^{a} is independently H, (C₁-C₈)alkyl, (C₂-C₈)alkenyl, (C₂-C₈)alkynyl, aryl(C₁-C₈)alkyl, (C₄-C₈)carbocyclylalkyl, —C(═O)R¹¹, —C(═O)OR¹¹, —C(═O)NR¹¹R¹², —C(═O)SR¹¹, —S(O)R¹¹, —S(O)₂R¹¹, —S(O)₂(OR¹¹), —S(O)₂(OR¹¹), or —SO₂NR¹¹R¹²;
R⁷ is H, —C(═O)R¹¹, —C(═O)OR¹¹, —C(O)NR¹¹R¹², —C(═O)SR¹¹, —S(O)R¹¹, —S(O)₂R¹¹, —S(O)(OR¹¹), —S(O)₂(OR¹¹), —SO₂NR¹¹R¹², or

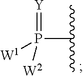

Y is O, S, NR, ⁺N(O)(R), N(OR), ⁺N(O)(OR), or N—NR₂;
W¹ and W², when taken together, are —Y³(C(R^{y})₂)₃Y³—;
or one of W¹ or W² together with either R³ or R⁴ is —Y³— and the other of W¹ or W² is Formula Ia;
or W¹ and W² are each, independently, a group of the Formula Ia:

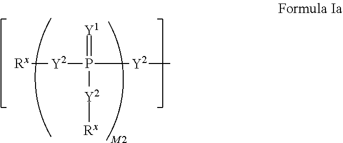

wherein:
each Y¹ is, independently, O, S, NR, ⁺N(O)(R), N(OR), ⁺N(O)(OR), or N—NR₂;
each Y² is independently a bond, O, CR₂, NR, ⁺N(O)(R), N(OR), ⁺N(O)(OR), N—NR₂, S, S—S, S(O), or S(O)₂;
each Y³ is independently O, S, or NR;
M2 is 0, 1 or 2;
each R^{x} is a group of the formula:

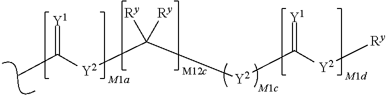

wherein:
each M1a, M1c, and M1d is independently 0 or 1;
M12c is 0, 1, 2, 3, 4, 5, 6, 7, 8, 9, 10, 11 or 12;
each R^{y} is independently H, F, Cl, Br, I, OH, —CN, —N₃, —NO₂, —OR, —C(═Y¹)R, —C(Y¹)W⁵, —C(═Y¹)OR, —C(═Y¹)N(R)₂, —N(R)₂, —⁺N(R)₃, —SR, —S(O)R, —S(O)₂R, —SO₂W⁵, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y)(N(R)₂), —N(R)C(═Y¹)R, —N(R)C(═Y¹)OR, —N(R)C(═Y¹)N(R)₂, —SO₂NR₂, W⁵, (C₁-C₈) alkyl, (C₁-C₈) substituted alkyl, (C₂-C₈)alkenyl, (C₂-C₈) substituted alkenyl, (C₂-C₈) alkynyl, (C₂-C₈) substituted alkynyl, C₆-C₂₀ aryl, C₆-C₂₀ substituted aryl, C₂-C₂₀ heterocyclyl, C₂-C₂₀ substituted heterocyclyl, arylalkyl or substituted arylalkyl; wherein each alkyl, alkenyl, alkynyl, aryl, heterocyclyl, or arylalkyl is independently optionally substituted with one or more Z groups;
or when taken together, two R^{y} on the same carbon atom form a carbocyclic ring of 3 to 7 carbon atoms;
each W⁵ is independently a carbocycle or a heterocycle optionally substituted with 1 to 3 R^{Z} groups;
each R^{Z} is independently F, Cl, Br, I, OH, —CN, —N₃, —NO₂, —OR, —C(═Y¹)R, —C(═Y¹)OR, —C(═Y¹)N(R)₂, —N(R)₂, —⁺N(R)₃, —SR, —S(O)R, —S(O)₂R, —S(O)(OR), —S(O)₂(OR), —OC(═Y¹)R, —OC(═Y¹)OR, —OC(═Y¹)(N(R)₂), —SC(═Y¹)R, —SC(═Y¹)OR, —SC(═Y)(N(R)₂), —N(R)C(═Y¹)R, —N(R)C(═Y¹)OR, —N(R)C(═Y)N(R)₂, —SO₂NR₂, (C₁-C₈) alkyl, (C₁-C₈) substituted alkyl, (C₂-C₈)alkenyl, (C₂-C₈) substituted alkenyl, (C₂-C₈) alkynyl, (C₂-C₈) substituted alkynyl, C₆-C₂₀ aryl, C₆-C₂₀ substituted aryl, C₂-C₂₀ heterocyclyl, C₂-C₂₀ substituted heterocyclyl, arylalkyl or substituted arylalkyl; wherein each alkyl, alkenyl, alkynyl, aryl, heterocyclyl, or arylalkyl is independently optionally substituted with one or more Z groups;
each R is independently H, (C₁-C₈) alkyl, (C₁-C₈) substituted alkyl, (C₂-C₈)alkenyl, (C₂-C₈) substituted alkenyl, (C₂-C₈) alkynyl, (C₂-C₈) substituted alkynyl, C₆-C₂₀ aryl, C₆-C₂₀ substituted aryl, C₂-C₂₀ heterocyclyl, C₂-C₂₀ substituted heterocyclyl, arylalkyl or substituted arylalkyl; wherein each alkyl, alkenyl, alkynyl, aryl, heterocyclyl, or arylalkyl is independently optionally substituted with one or more Z groups;
each X¹ or X² is independently C—R¹⁰ or N;
each R⁸ is halogen, NR¹¹R¹², N(R¹¹)OR¹¹, NR¹¹NR¹¹R¹², N₃, NO, NO₂, CHO, CN, —CH(═NR¹¹), —CN═NHNR¹¹, —CN═N(OR¹¹), —CH(OR¹¹)₂, —C(═O)NR¹¹R¹², —C(═S)NR¹¹R¹², —C(═O)OR¹¹, (C₁-C₈)alkyl, (C₂-C₈)alkenyl, (C₂-C₈)alkynyl, (C₄-C₈)carbocyclylalkyl, optionally substituted aryl, optionally substituted heteroaryl, —C(═O)(C₁-C₈)alkyl, —S(O)ₙ(C₁-C₈)alkyl, aryl(C₁-C₈)alkyl, OR¹¹ or SR¹¹; wherein each aryl or heteroaryl is independently optionally substituted with one or more Z groups;
each R⁹ or R¹⁰ is independently H, halogen, NR¹¹R¹², N(R¹¹)OR¹¹, NR¹¹NR¹¹R¹², N₃, NO, NO₂, CHO, CN, —CH(═NR¹¹), —CN═NHNR¹¹, —CN═N(OR¹), —CH(OR¹¹)₂, —C(═O)NR¹¹R¹², —C(═S)NR¹¹R¹², —C(═O)OR¹¹, R¹¹, OR¹¹ or SR¹¹;
each R¹¹ or R¹² is independently H, (C₁-C₈)alkyl, (C₂-C₈)alkenyl, (C₂-C₈)alkynyl, (C₄-C₈)carbocyclylalkyl, optionally substituted aryl, optionally substituted heteroaryl, —C(═O)(C₁-C₈)alkyl, —S(O)ₙ(C₁-C₈)alkyl or aryl(C₁-C₈)alkyl; wherein each aryl or heteroaryl is independently optionally substituted with one or more Z groups;
or R¹¹ and R¹² taken together with a nitrogen to which they are both attached form a 3 to 7 membered heterocyclic ring wherein any one carbon atom of said heterocyclic ring can optionally be replaced with —O—, —S— or —NR^{a}—;
each Z is independently halogen. —O⁻, ═O, —OR^{b}, —SR^{b}, —S⁻, —NR^{b} ₂, —N⁺R^{b}₃, ═NR^{b}, —CN, —OCN, —SCN, —N═C═O, —NCS, —NO, —NO₂, ═N₂, —N₃, —NHC(═O)R^{b}, —OC(═O)R^{b}, —NHC(═O)NR^{b}₂, —S(═O)₂—, —S(═O)₂OH, —S(═O)₂R^{b}, —OS(═O)₂OR^{b}, —S(═O)₂NR^{b}₂, —S(═O)R^{b}, —OP(═O)(OR^{b})₂, —P(═O)(OR^{b})₂, —P(═O)(O⁻)₂, —P(═O)(OH)₂, —P(O)(OR^{b})(O—), —C(═O)R^{b}, —C(═O)X, —C(S)R^{b}, —C(O)OR^{b}, —C(O)O—, —C(S)OR^{b}, —C(O)SR^{b}, —C(S)SR^{b}, —C(O)NR^{b} 2, —C(S)NR^{b} 2, —C(═NR^{b})NR^{b}₂, where each R^{b} is independently H, alkyl, aryl, arylalkyl, or heterocycle;
wherein each (C₁-C₈)alkyl, (C₂-C₈)alkenyl, (C₂-C₈)alkynyl or aryl(C₁-C₈)alkyl of each R¹, R², R³, R⁴, R⁵, R⁶, R¹¹ or R¹² is, independently, optionally substituted with one or more halo, hydroxy, CN, N₃, N(R^{a})₂ or OR^{a}; and wherein one or more of the non-terminal carbon atoms of each said (C₁-C₈)alkyl is optionally replaced with —O—, —S— or —NR^{a}—.

In some embodiments, the provided compound is a compound for Formula IV:

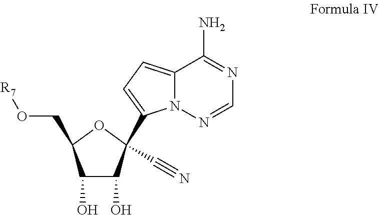

or a pharmaceutically acceptable salt, hydrate or ester, thereof;
wherein,
R⁷ is selected from the group consisting of
a) H, —C(═O)R¹¹, —C(═O)OR¹¹, —C(═O)NR¹¹R¹², —C(═O)SR¹¹, —S(O)R¹¹, —S(O)₂R¹¹, —S(O)₂(OR¹¹), or —SO₂NR¹¹R¹²;
b)

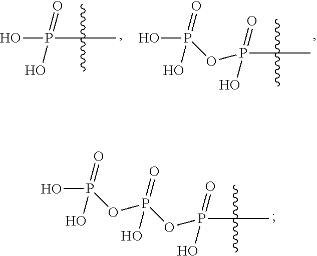 [SMILES: *P(=O)(O)O ; *P(=O)(O)OP(=O)(O)O ; *P(=O)(O)OP(=O)(O)OP(=O)(O)O]

c) a group selected from:

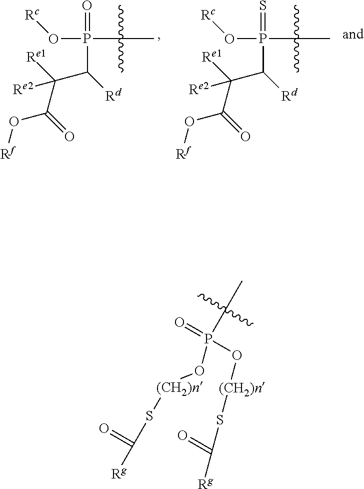

wherein:
R^{c} is selected from the group of phenyl, 1-naphthyl, 2-naphthyl,

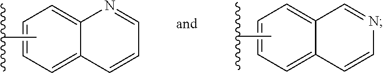

R^{d} is selected from the group of H or CH₃;
R^{e1} and R^{e2} are each independently selected from the group of H, (C₁-C₆)alkyl or benzyl;
R¹ is selected from the group of from H, (C₁-C₈)alkyl, benzyl, (C₃-C₆)cycloalkyl, and —CH₂—(C₃₋₆)cycloalkyl;
R⁹ is selected from selected from the group of (C₁-C₈)alkyl, —O—(C₁-C₈)alkyl, benzyl, —O-benzyl, —CH₂—(C₃-C₆)cycloalkyl, —O—CH₂—(C₃-C₆)cycloalkyl, and CF₃; and
n′ is an integer selected from the group of 1, 2, 3, and 4; and
d) a group of the formula:

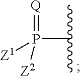

wherein:
Q is selected from the group of O, S, NR, +N(O)(R), N(OR), ⁺N(O)(OR), or N—NR₂;
Z¹ and Z², when taken together, are -Q¹(C(R^{y})₂)₃Q¹-;
wherein
each Q¹ is independently selected from the group of O, S, or NR; and
each R^{y} is independently selected from the group of H, F, Cl, Br, I, OH, R, —C(=Q²)R, —C(=Q²)OR, —C(=Q²)N(R)₂, —N(R)₂, —⁺N(R)₃, —SR, —S(O)R, —S(O)₂R, —S(O)(OR), —S(O)₂(OR), —OC(=Q¹)R, —OC(=Q²)OR, —OC(=Q²)(N(R)₂), —SC(=Q²)R, —SC(=Q²)OR, —SC(=Q²)(N(R)₂), —N(R)C(=Q²)R, —N(R)C(=Q²)OR, —N(R)C(=Q²)N(R)₂, —SO₂NR₂, —CN, —N₃, —NO₂, —OR, or Z³; or when taken together, two R^{y} on the same carbon atom form a carbocyclic ring of 3 to 7 carbon atoms;
each Q² is independently, O, S, NR, ⁺N(O)(R), N(OR), ⁺N(O)(OR), or N—NR₂; or
Z¹ and Z² are each, independently, a group of the Formula Ia:

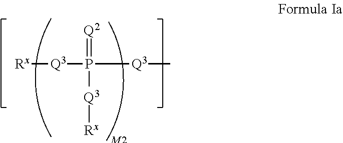

wherein:
each Q³ is independently selected from the group of a bond, O, CR₂, NR, ⁺N(O)(R), N(OR), ⁺N(O)(OR), N—NR₂, S, S—S, S(O), or S(O)₂;
M2 is an integer selected from the group of 0, 1 or 2;
each R^{x} is independently R^{y} or the formula:

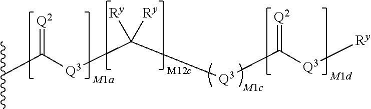

wherein:
each M1a, M1c, and M1d is an integer independently selected from the group of 0 or 1;
M12c is an integer selected from the group of 0, 1, 2, 3, 4, 5, 6, 7, 8, 9, 10, 11 or 12;
Z³ is Z⁴ or Z⁵;
Z⁴ is R, —C(Q²)R^{Y}, —C(Q²)Z⁵, —SO₂R^{y}, or —SO₂Z⁵; and
Z⁵ is a carbocycle or a heterocycle wherein Z⁵ is independently substituted with 0 to 3 R^{y} groups;
each R¹¹ or R¹² is independently H, (C₁-C₈)alkyl, (C₂-C₈)alkenyl, (C₂-C₈)alkynyl, (C₄-C₈)carbocyclylalkyl, (C₆-C₂₀)optionally substituted aryl, optionally substituted heteroaryl, —C(═O)(C₁-C₈)alkyl, —S(O)ₙ(C₁-C₈)alkyl or (C₆-C₂₀)aryl(C₁-C₈)alkyl; or R¹ and R¹² taken together with a nitrogen to which they are both attached form a 3 to 7 membered heterocyclic ring wherein any one carbon atom of said heterocyclic ring can optionally be replaced with —O—, —S— or —NR^{a}—;
each R^{a} is independently selected from the group of H, (C₁-C₈)alkyl, (C₂-C₈)alkenyl, (C₂-C₈)alkynyl, (C₆-C₂₀)aryl(C₁-C₈)alkyl, (C₄-C₈)carbocyclylalkyl, —C(═O)R, —C(═O)OR, —C(═O)NR₂, —C(═O)SR, —S(O)R, —S(O)₂R, —S(O)(OR), —S(O)₂(OR), or —SO₂NR₂; wherein
each R is independently selected from the group of H, (C₁-C₈) alkyl, (C₁-C₈) substituted alkyl, (C₂-C₈)alkenyl, (C₂-C₈) substituted alkenyl, (C₂-C₈) alkynyl, (C₂-C₈) substituted alkynyl, (C₆-C₂₀)aryl, (C₆-C₂₀)substituted aryl, (C₂-C₂₀)heterocyclyl, (C₂-C₂₀)substituted heterocyclyl, (C₆-C₂₀)aryl(C₁-C₈)alkyl or substituted (C₆-C₂₀)aryl(C₁-C₈)alkyl;
each n is an integer independently selected from the group of 0, 1, or 2; and wherein each (C₁-C₈)alkyl, (C₂-C₈)alkenyl, (C₂-C₈)alkynyl or (C₆-C₂₀)aryl(C₁-C₈)alkyl of each R¹¹ or R¹² is, independently, optionally substituted with one or more substituents selected from the group of halo, hydroxy, CN, N₃, N(R^{a})₂ or OR^{a}; and wherein one or more of the non-terminal carbon atoms of each said (C₁-C₈)alkyl may be optionally replaced with —O—, —S— or —NR^{a}.

In one embodiment, the nucleoside analog and/or viral RNA polymerase inhibitor or prodrug thereof is Remdesivir:

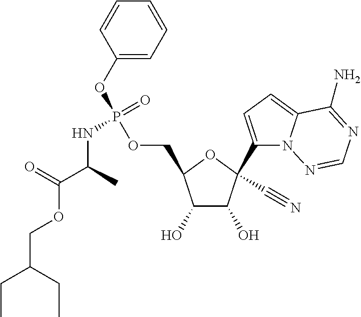 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

or a pharmaceutically acceptable salt thereof.

In another embodiment, the nucleoside analog and/or viral RNA polymerase inhibitor or prodrug thereof is GS-441524:

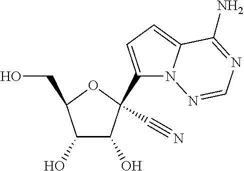 [SMILES: N#C[C@@]1(c2ccc3c(N)ncnn23)O[C@H](CO)[C@@H](O)[C@H]1O]

or a pharmaceutically acceptable salt thereof.

In another embodiment, the nucleoside analog and/or viral RNA polymerase inhibitor or prodrug thereof is Molnupiravir (MK-4482, EIDD-2801), also known as β-D-N4-hydroxycytidine:

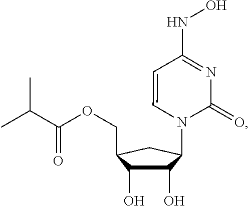

or a pharmaceutically acceptable salt thereof.

In another embodiment, the nucleoside analog and/or viral RNA polymerase inhibitor or prodrug thereof is tenofovir, lamivudine, entecavir, sofosbuvir, acyclovir. idoxuridine, edoxudine, trifluridine, vidarabine, brivudine, acyclovir, ganciclovir, valaciclovir, cidofovir, valganciclovir, penciclovir, famciclovir, zidovudine, didanosine, zalcitabine, stavudine, abacavir, emtricitabine, adefovir, telbivudine, and/or ribavirin.

### Combinations

The inventors of the present application have invented combinations of compositions exhibiting surprising and significant synergistic effects. In particular, a combination of a viral entry inhibitor and a nucleoside analog/RNA polymerase inhibitor exhibits surprising and significant synergistic effects, including a combination of K11777 and Remdesivir or K11777 and GS-441524.

### Synthesis

In some embodiments, the compounds of the present application may be synthesized as described in Jaishankar P, Hansell E, Zhao D M, Doyle P S, McKerrow J H, et al. Potency and selectivity of P2/P3-modified inhibitors of cysteine proteases from trypanosomes. Bioorg Med Chem Lett. 2008; 18:624-628. In some embodiments, the compounds of the present application may also be prepared as described in Chen et al, In Vitro and In Vivo Studies of the Trypanocidal Properties of WRR-483 against Trypanosoma cruzi, DOI: 10.1371/journal.pntd.0000825. Camostat is available commercially, e.g. from Sigma Aldrich. In some embodiments, the compound of the present application may be prepared as discussed in U.S. Pat. No. 8,008,264 or 9,724,360. In other embodiments, a person of ordinary skill will be knowledgeable regarding standard synthesis routes to certain embodiments of the compounds of the present application.

### Formulations

In some embodiments, compounds of the present application and/or combinations thereof may be formulated as a solid oral dose. In particular, such solid oral dose may be a capsule or a tablet. In some embodiments, the solid oral dose may comprise microspheres that can be sprinkled in liquid or food. Such microspheres may be preferable as dosage forms for certain patient subpopulations that are otherwise unable to ingest capsule or tablet forms.

In some embodiments, compounds of the present application and/or combinations thereof may be formulated in a liquid suspension or emulsion. In some embodiments, formulations of the present application may include solubilizers, stabilizers, buffers, tonicity modifiers, bulking agents, viscosity enhancers/reducers, surfactants, chelating agents, and/or adjuvants.

In some embodiments, compounds of the present application and/or combinations thereof may be formulated as an intravenous injection. In some embodiments, compounds of the present application and/or combinations thereof may be formulated as an inhalable.

In some embodiments, compounds of the present application are formulated as HCl and/or mesylate salts. One embodiment of the present application, for example, provides K11777 HCl. Another embodiment of the present application provides camostat mesylate. In some embodiments, an HCl salt is neutralized in solution.

In some embodiments, compounds of the present application are prodrugs. For example, and without being bound by theory, Remdesivir is a prodrug that is intended to allow intracellular delivery of GS-441524 monophosphate and subsequent biotransformation into GS-441524 triphosphate, a ribonucleotide analogue inhibitor of viral RNA polymerase.

Methods of Administration and/or Dosing

In some embodiments, compounds of the present application and/or combinations thereof are dosed to a subject in need thereof for the prevention and/or treatment of a disease. Thus, in one aspect of the present application, the antiviral compounds, combinations, formulations, and methods of the present application are useful for treating viral infections, including viral infections which rely on activation by cellular proteases and viruses capable of incorporating nucleoside analogs (like Remdesivir and GS-441524) during replication. Such infections may be caused by Filoviruses (e.g., Ebola virus (EBOV) and Marburg virus (MARV)), Coronaviruses (e.g., SARS-CoV-2, SARS-CoV, MERS-CoV), Influenza viruses (e.g., Influenzavirus A (H1N1, H7N9, H5N1, etc.), B, C, and D), Hepatitis viruses (e.g. Hepatitis A, B, C, D, E), Retroviruses (e.g., Lentiviruses including HIV), Herpesviruses (e.g. Varicella Zoster, Herpes simplex 1 and 2, etc.), Rhinoviruses, Rotaviruses, Human Papillomaviruses, Henipaviruses, Adenoviruses, Noroviruses, Rotaviruses, Sapoviruses, Astroviruses, Coxsackieviruses, Echoviruses, Enteroviruses, Orthomyxoviruses, Paramyxoviruses (e.g., Measles virus), Motonaviruses (e.g. Rubella viruses), Flaviviruses (e.g. Dengue viruses, Yellow fever viruses), Poxviruses (e.g., Variola virus), Alphaviruses (e.g., Chikungunya virus), Orthohantaviruses (e.g. Hanta virus), and Rabies viruses.

In some embodiments, compounds of the present application and/or combinations thereof are administered to treat one of the foregoing. In some embodiments, compounds of the present application and/or combinations thereof are administered to delay the onset of one of the foregoing. In some embodiments, compounds of the present application and/or combinations thereof are administered to prevent one of the foregoing. In some embodiments, compounds of the present application and/or combinations thereof are administered to attenuate one of the foregoing.

In some embodiments, compounds of the present application and/or combinations thereof are dosed at approximately 0.5 mg/kg. In some embodiments, compounds of the present application and/or combinations thereof are dosed at approximately 1 mg/kg. In some embodiments, compounds of the present application and/or combinations thereof are dosed at approximately 2 mg/kg. In some embodiments, compounds of the present application and/or combinations thereof are dosed at approximately 3 mg/kg. In some embodiments, compounds of the present application and/or combinations thereof are dosed at approximately 4 mg/kg. In some embodiments, compounds of the present application and/or combinations thereof are dosed at approximately 5 mg/kg. In some embodiments, compounds of the present application and/or combinations thereof are dosed at approximately 6 mg/kg. In some embodiments, compounds of the present application and/or combinations thereof are dosed at approximately 7 mg/kg. In some embodiments, compounds of the present application and/or combinations thereof are dosed at approximately 8 mg/kg. In some embodiments, compounds of the present application and/or combinations thereof are dosed at approximately 9 mg/kg. In some embodiments, compounds of the present application and/or combinations thereof are dosed at approximately 10 mg/kg.

In some embodiments, compounds of the present application and/or combinations thereof are dosed at approximately 10 mg/kg. In some embodiments, compounds of the present application and/or combinations thereof are dosed at approximately 20 mg/kg. In some embodiments, compounds of the present application and/or combinations thereof are dosed at approximately 30 mg/kg. In some embodiments, compounds of the present application and/or combinations thereof are dosed at approximately 40 mg/kg. In some embodiments, compounds of the present application and/or combinations thereof are dosed at approximately 50 mg/kg. In some embodiments, compounds of the present application and/or combinations thereof are dosed at approximately 60 mg/kg. In some embodiments, compounds of the present application and/or combinations thereof are dosed at approximately 70 mg/kg. In some embodiments, compounds of the present application and/or combinations thereof are dosed at approximately 80 mg/kg. In some embodiments, compounds of the present application and/or combinations thereof are dosed at approximately 90 mg/kg. In some embodiments, compounds of the present application and/or combinations thereof are dosed at approximately 100 mg/kg.

In some embodiments, compounds of the present application and/or combinations thereof are dosed at approximately 50 mg/day qd, bid, or tid. In some embodiments, compounds of the present application and/or combinations thereof are dosed at approximately 100 mg/day qd, bid, or tid. In some embodiments, compounds of the present application and/or combinations thereof are dosed at approximately 150 mg/day qd, bid, or tid. In some embodiments, compounds of the present application and/or combinations thereof are dosed at approximately 200 mg/day qd, bid, or tid. In some embodiments, compounds of the present application and/or combinations thereof are dosed at approximately 250 mg/day qd, bid, or tid. In some embodiments, compounds of the present application and/or combinations thereof are dosed at approximately 300 mg/day qd, bid, or tid. In some embodiments, compounds of the present application and/or combinations thereof are dosed at approximately 350 mg/day qd, bid, or tid. In some embodiments, compounds of the present application and/or combinations thereof are dosed at approximately 400 mg/day qd, bid, or tid. In some embodiments, compounds of the present application and/or combinations thereof are dosed at approximately 450 mg/day qd, bid, or tid. In some embodiments, compounds of the present application and/or combinations thereof are dosed at approximately 500 mg/day qd, bid, or tid.

In some embodiments, compounds of the present application and/or combinations thereof are dosed at approximately 1 g/day qd, bid, or tid. In some embodiments, compounds of the present application and/or combinations thereof are dosed at approximately 2 g/day qd, bid, or tid. In some embodiments, compounds of the present application and/or combinations thereof are dosed at approximately 3 g/day qd, bid, or tid. In some embodiments, compounds of the present application and/or combinations thereof are dosed at approximately 4 g/day qd, bid, or tid. In some embodiments, compounds of the present application and/or combinations thereof are dosed at approximately 5 g/day qd, bid, or tid.

In some embodiments, a treatment regimen for compounds of the present application and/or combinations thereof comprise administering said compounds or combinations for 5 to 14 days. In some embodiments, a treatment regimen for compounds of the present application and/or combinations thereof comprise administering said compounds or combinations for 5, 6, 7, 8, 9, 10, 11, 12, 13, or 14 days.

In some embodiments, a treatment regimen for compounds of the present application and/or combinations thereof comprise administering said compounds or combinations for 1 to 10 days. In some embodiments, a treatment regimen for compounds of the present application and/or combinations thereof comprise administering said compounds or combinations for 5 to 10 days. In some embodiments, a treatment regimen for compounds of the present application and/or combinations thereof comprise administering said compounds or combinations for 1 to 5 days. In some embodiments, a treatment regimen for compounds of the present application and/or combinations thereof comprise administering said compounds or combinations for 1 to 14 days. In some embodiments, a treatment regimen for compounds of the present application and/or combinations thereof comprise administering said compounds or combinations for 7 to 14 days. In some embodiments, a treatment regimen for compounds of the present application and/or combinations thereof comprise administering said compounds or combinations for 1 to 21 days. In some embodiments, a treatment regimen for compounds of the present application and/or combinations thereof comprise administering said compounds or combinations for 7 to 21 days. In some embodiments, a treatment regimen for compounds of the present application and/or combinations thereof comprise administering said compounds or combinations for 14 to 21 days.

In some embodiments, a treatment regimen for compounds of the present application and/or combinations thereof includes a titration phase followed by a maintenance phase. In some embodiments, in the titration phase a dose of the compound or the combination which is less than the dose administered during the maintenance phase is increased stepwise over a plurality of days up to the dose received at the maintenance phase. In some embodiments, the titration phase lasts 1 day, 2 days, 3 days, 4 days, or 5 days.

In some embodiments, a treatment regimen for compounds of the present application and/or combinations thereof includes a loading dose phase followed by a maintenance phase. In some embodiments, in the loading dose phase a dose of the compound or the combination which is greater than the maintenance dose is administered over a plurality of days. In some embodiments, the loading dose phase lasts 1 day, 2 days, 3 days, 4 days, or 5 days. In some embodiments, dosing during the loading dose phase is increased stepwise. In some embodiments, dosing during the loading dose phase is constant.

In some embodiments, the present application includes identifying patients at risk for side effects and/or complications associated with dosing the compounds, combinations, or pharmaceutical formulations according to the present application. In some embodiments, such patients include those receiving ACE inhibitors or ARBs. In some embodiments, the dosing is altered to reduce side effects and/or complications associated with dosing the compounds.

In some embodiments, compounds or combinations of the present application are administered to prevent or delay the onset of a disease. Thus, some methods of the present application include identifying a patient in need of prevention or delaying the onset of a disease. Preferably, methods of the present application include identifying a patient in need of prevention or delaying of the onset of viral infections, including viral infections which rely on activation by cellular proteases and viruses capable of incorporating nucleoside analogs (like Remdesivir and GS-441524) during replication. In some embodiments, the method includes screening for front-line healthcare workers and first responders. In some embodiments, the method includes screening for residents and staff of retirement communities. In some embodiments, the method includes screening for staff, teachers, and students attending educational facilities. In some embodiments, the method includes screening for staff and first responders who work in emergency rooms. In some embodiments, the method includes screening for military personnel. In some embodiments, the method includes administering a test for the presence of viral infections, including viral infections which rely on activation by cellular proteases and viruses capable of incorporating nucleoside analogs (like Remdesivir and GS-441524) during replication, in a human. Such test may include a PCR-based assay for identifying viral RNA fragments associated with Filoviruses (e.g., Ebola virus (EBOV) and Marburg virus (MARV)), Coronaviruses (e.g., SARS-CoV-2, SARS-CoV, MERS-CoV), Influenza viruses (e.g., Influenzavirus A (H1N1, H7N9, H5N1, etc.), B, C, and D), Hepatitis viruses (e.g. Hepatitis A, B, C, D, E), Retroviruses (e.g., Lentiviruses including HIV), Herpesviruses (e.g. Varicella Zoster, Herpes simplex 1 and 2, etc.), Rhinoviruses, Rotaviruses, Human Papillomaviruses, Henipaviruses, Adenoviruses, Noroviruses, Rotaviruses, Sapoviruses, Astroviruses, Coxsackieviruses, Echoviruses, Enteroviruses, Orthomyxoviruses, Paramyxoviruses (e.g., Measles virus), Motonaviruses (e.g. Rubella viruses), Flaviviruses (e.g. Dengue viruses, Yellow fever viruses), Poxviruses (e.g., Variola virus), Alphaviruses (e.g., Chikungunya virus), Orthohantaviruses (e.g. Hanta virus), and Rabies viruses.

Such test may also include enzyme-linked immunosorbent assay (ELISA) or a variant thereof, including colorimetric, fluorescent, or luminescent ELISA variants such as fluorescent colorimetric immunoassay (CIA), immunoassay (FIA), or chemiluminescent immunoassay (CLIA). In comparison to PCR-based approaches, ELISA based approaches present results rapidly. Further in comparison to PCR-based approaches, certain ELISA based approaches, including colorimetric, fluorescent, or luminescent ELISA variants present results which are both rapid and easy to read.

In some embodiments, the treatment regimen includes administration of one or more formulations of the compounds or combinations of the present application. Thus, in some embodiments, the methods include administering a solid oral dose, e.g., a capsule, a tablet, or microspheres, a liquid suspension or emulsion, an intravenous injection. In some embodiments, compounds of the present application and/or combinations thereof may be formulated as an inhalable.

### Kits

Embodiments of the present application includes kits containing compounds, combinations, and formulations according to the present application. In some embodiments, the kits include instructions for administering embodiments of the present application. In some embodiments, the kits include a test for the presence of viral infections, including viral infections which rely on activation by cellular proteases and viruses capable of incorporating nucleoside analogs (like Remdesivir and GS-441524) during replication, in a human. Such test may include a PCR-based assay for identifying viral RNA fragments associated with Filoviruses (e.g., Ebola virus (EBOV) and Marburg virus (MARV)), Coronaviruses (e.g., SARS-CoV-2, SARS-CoV, MERS-CoV), Influenza viruses (e.g., Influenzavirus A (H1N1, H7N9, H5N1, etc.), B, C, and D), Hepatitis viruses (e.g. Hepatitis A, B, C, D, E), Retroviruses (e.g., Lentiviruses including HIV), Herpesviruses (e.g. Varicella Zoster, Herpes simplex 1 and 2, etc.), Rhinoviruses, Rotaviruses, Human Papillomaviruses, Henipaviruses, Adenoviruses, Noroviruses, Rotaviruses, Sapoviruses, Astroviruses, Coxsackieviruses, Echoviruses, Enteroviruses, Orthomyxoviruses, Paramyxoviruses (e.g., Measles virus), Motonaviruses (e.g. Rubella viruses), Flaviviruses (e.g. Dengue viruses, Yellow fever viruses), Poxviruses (e.g., Variola virus), Alphaviruses (e.g., Chikungunya virus), Orthohantaviruses (e.g. Hanta virus), and Rabies viruses.

Such test may also include enzyme-linked immunosorbent assay (ELISA) or a variant thereof, including colorimetric, fluorescent, or luminescent ELISA variants such as fluorescent colorimetric immunoassay (CIA), immunoassay (FIA), or chemiluminescent immunoassay (CLIA). In comparison to PCR-based approaches, ELISA based approaches present results rapidly. Further in comparison to PCR-based approaches, certain ELISA based approaches, including colorimetric, fluorescent, or luminescent ELISA variants present results which are both rapid and easy to read. Thus, the kits of the present application are useful to treat disease in certain areas where rapid testing is advantageous or where testing by individuals with minimal training may be necessary, including, but not limited to, remote and rural areas, Indian reservations, locations with insufficient medical services to timely administer testing, locations associated with military presence, such as remote military bases or areas in which active combat is likely or is currently taking place.

In some embodiments, the kits of the present application include individually packaged formulations for compounds and/or combinations of the present application, for example individually packaged capsules or tablets. In some embodiments, the kits of the present application include liquid formulations, optionally for direct administration or for mixing and/or titration by a medical professional. In some embodiments, the kits of the present application include an inhaler or similar device for inhalation of an inhalable formulation.

In some embodiments, the kits of the present application include a contact tracing questionnaire or incident report. In some embodiments, such contact tracing questionnaire or incident report is in paper format, and in other embodiments such contact tracing questionnaire or incident report is in electronic format, e.g., utilizing a hyperlink or a QR code directing to a web application or an app-based implementation.

### EXAMPLES

### Example 1—Clinical Trial

A randomized, double-blind, placebo-controlled trial to evaluate the safety and efficacy of novel therapeutic agents in hospitalized adults diagnosed with a viral infection.
Enrollment: 100-500 patients.
Intervention:
   Combination of K11777 (250 mg/day) and Remdesivir (200 mg/day followed by maintenance dose of 100 mg/day) in capsule and/or injectable formulations.
   Combination of K11777 (250 mg/day) and Remdesivir (100 mg/day) in capsule and/or injectable formulations.
   Placebo
Primary outcome measure:
   Percentage of subjects reporting each severity rating on an 8-point ordinal scale [Time Frame: Day 15]
      The ordinal scale is an assessment of the clinical status at the first assessment of a given study day. The scale is as follows:
         1. Death;
         2. Hospitalized, on invasive mechanical ventilation or extracorporeal membrane oxygenation (ECMO);
         3. Hospitalized, on non-invasive ventilation or high flow oxygen devices;
         4. Hospitalized, requiring supplemental oxygen;
         5. Hospitalized, not requiring supplemental oxygen—requiring ongoing medical care;
         6. Hospitalized, not requiring supplemental oxygen—no longer requires ongoing medical care;
         7. Not hospitalized, limitation on activities and/or requiring home oxygen;
         8. Not hospitalized, no limitations on activities.
Secondary outcome measures:
   Blood chemistry (liver transaminases, serum creatinine, glucose, hemoglobin, etc.)
   Clinical status using ordinal scale
   Cumulative Serious Adverse Events (SAEs)
   Discontinuation or temporary cessation of treatment
   Duration of hospitalization
   Incidence and duration of non-invasive ventilation
   Incidence and duration of new oxygen use
   Incidence and duration of new ventilator or extracorporeal membrane oxygenation (ECMO) use
   Mean change in the ordinal scale
   Number of non-invasive ventilation/high flow oxygen free days
   Number of oxygenation free days
   Ventilator/extracorporeal membrane oxygenation (ECMO) free days
   Subject mortality
      Time to an improvement of one category using an ordinal scale

1. A method of treating a viral infection, the method comprising:
administering a pharmaceutical composition comprising
a viral entry inhibitor and
a nucleoside analog or RNA polymerase inhibitor, or prodrug thereof.

2. The method of claim 1, wherein the viral entry inhibitor is K11777 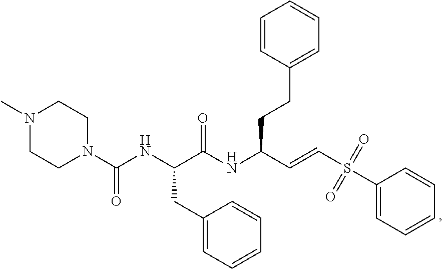 [SMILES: CN1CCN(C(=O)N[C@@H](Cc2ccccc2)C(=O)N[C@H](/C=C/S(=O)(=O)c2ccccc2)CCc2ccccc2)CC1]
or a pharmaceutically acceptable salt thereof.

3. The method of claim 1, wherein the nucleoside analog or RNA polymerase inhibitor, or prodrug thereof is Remdesivir 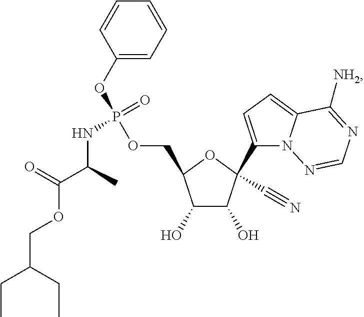 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]
or a pharmaceutically acceptable salt thereof.

4. The method of claim 1, wherein the viral infection is caused by a virus selecting from the group consisting of Filoviruses, Coronaviruses, Influenza viruses, Hepatitis viruses, Retroviruses, Herpesviruses, Rhinoviruses, Rotaviruses, Human Papillomaviruses, Henipaviruses, Adenoviruses, Noroviruses, Rotaviruses, Sapoviruses, Astroviruses, Coxsackieviruses, Echoviruses, Enteroviruses, Orthomyxoviruses, Paramyxoviruses Motonaviruses, Flaviviruses, Poxviruses, Alphaviruses, Orthohantaviruses, and Rabies viruses.

5. The method of claim 4, wherein the viral infection is caused by a Filovirus.

6. The method of claim 5, wherein the viral infection is caused by Ebola virus or Marburg virus.

7. The method of claim 4, wherein the viral infection is caused by an Influenza virus.

8. The method of claim 4, wherein the viral infection is caused by a Hepatitis virus.

9. The method of claim 4, wherein the viral infection is caused by a Retrovirus.

10. The method of claim 4, wherein the viral infection is caused by a Herpes virus.

11. A method of preventing or delaying the onset of a viral infection in a subject in need thereof, the method comprising:
administering a pharmaceutical composition comprising
a viral entry inhibitor and
a nucleoside analog or RNA polymerase inhibitor, or prodrug thereof.

12. The method of claim 11, wherein the viral entry inhibitor is K11777 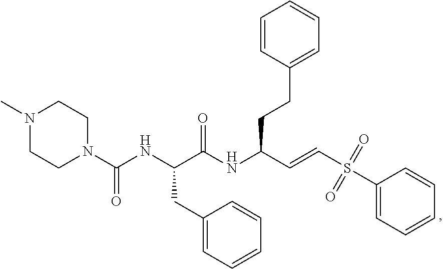 [SMILES: CN1CCN(C(=O)N[C@@H](Cc2ccccc2)C(=O)N[C@H](/C=C/S(=O)(=O)c2ccccc2)CCc2ccccc2)CC1]
or a pharmaceutically acceptable salt thereof.

13. The method of claim 11, wherein the nucleoside analog or RNA polymerase inhibitor, or prodrug thereof is Remdesivir 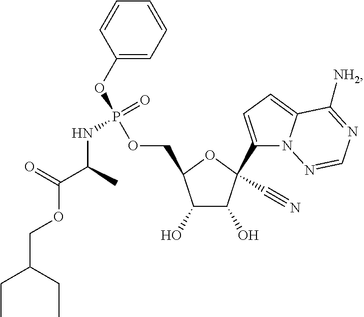 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]
or a pharmaceutically acceptable salt thereof.

14. The method of claim 11, wherein the viral infection is caused by a virus selecting from the group consisting of Filoviruses, Coronaviruses, Influenza viruses, Hepatitis viruses, Retroviruses, Herpesviruses, Rhinoviruses, Rotaviruses, Human Papillomaviruses, Henipaviruses, Adenoviruses, Noroviruses, Rotaviruses, Sapoviruses, Astroviruses, Coxsackieviruses, Echoviruses, Enteroviruses, Orthomyxoviruses, Paramyxoviruses Motonaviruses, Flaviviruses, Poxviruses, Alphaviruses, Orthohantaviruses, and Rabies viruses.

15. The method of claim 14, wherein the viral infection is caused by a Filovirus.

16. The method of claim 15, wherein the viral infection is caused by Ebola virus or Marburg virus.

17. The method of claim 14, wherein the viral infection is caused by an Influenza virus.

18. The method of claim 14, wherein the viral infection is caused by a Hepatitis virus.

19. The method of claim 14, wherein the viral infection is caused by a Retrovirus.

20. The method of claim 14, wherein the viral infection is caused by a Herpes virus.

21. A pharmaceutical composition comprising:
a viral entry inhibitor and
a nucleoside analog or RNA polymerase inhibitor or prodrug thereof.

22. The pharmaceutical composition according to claim 21, wherein the viral entry inhibitor is K11777 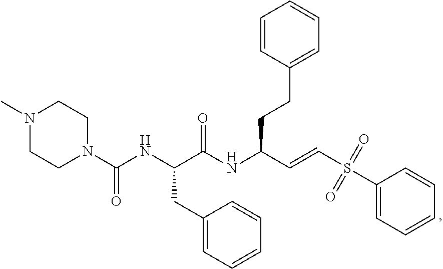 [SMILES: CN1CCN(C(=O)N[C@@H](Cc2ccccc2)C(=O)N[C@H](/C=C/S(=O)(=O)c2ccccc2)CCc2ccccc2)CC1]
or a pharmaceutically acceptable salt thereof.

23. The pharmaceutical composition according to claim 21, wherein the nucleoside analog or RNA polymerase inhibitor or prodrug thereof is Remdesivir 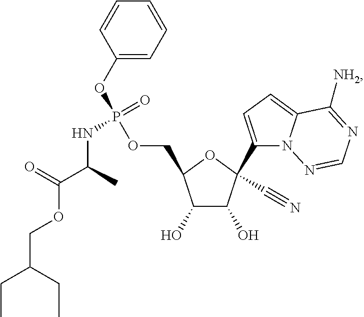 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]
or a pharmaceutically acceptable salt thereof.

24. The pharmaceutical composition according to claim 21, wherein the nucleoside analog or RNA polymerase inhibitor or prodrug thereof is GS-441524 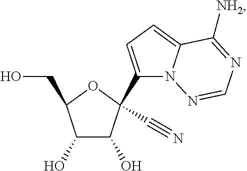 [SMILES: N#C[C@@]1(c2ccc3c(N)ncnn23)O[C@H](CO)[C@@H](O)[C@H]1O]
or a pharmaceutically acceptable salt thereof.

25. The pharmaceutical composition according to claim 21, wherein the nucleoside analog or RNA polymerase inhibitor or prodrug thereof is Molnupiravir: 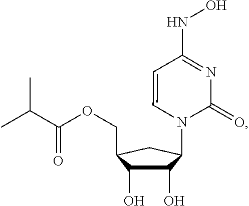
or a pharmaceutically acceptable salt thereof.
